(19) United States
(12) Patent Application Publication
(10) Pub. No.: US 2023/0255661 A1
(43) Pub. Date: Aug. 17, 2023

(54) Skin Nicking Device for Catheter Placement System

(71) Applicant: Bard Access Systems, Inc., Salt Lake City, UT (US)

(72) Inventor: Glade H. Howell, Draper, UT (US)

(21) Appl. No.: 18/109,807

(22) Filed: Feb. 14, 2023

Related U.S. Application Data
(60) Provisional application No. 63/310,497, filed on Feb. 15, 2022.

(51) Int. Cl. A61B 17/3209; A61B 17/34

(52) U.S. Cl. CPC A61B 17/32093; A61B 17/3415; A61B 2017/00336

(57) ABSTRACT

A catheter placement system having a catheter and a skin nicking device that includes a blade configured to nick the skin of a patient adjacent a catheter insertion site. The catheter includes a multi-luminal catheter tube coupled with multiple extension legs. The catheter tube includes a diameter transitioning section disposed between two sections of the catheter tube having different diameters. The skin nicking device is disposed within a lumen such that a blade at the distal of the skin nicking device protrudes from an aperture of the lumen. The skin nicking device is positionable and rotatable within lumen and removable from the lumen. The skin nicking device includes an elongate body that may include rigid portions and/or flexible portions.

### PRIORITY

This application claims the benefit of priority to U.S. Provisional Application No. 63/310,497, filed Feb. 15, 2022, which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma or increased risk of infection. There is a relatively high potential for touch contamination due to the number of medical devices that need to be interchanged during the Seldinger technique. As such, advanced catheter placement systems have been developed to reduce the number of steps and medical devices involved in placing a catheter, such as a CVC, into a patient.

Some of these advanced catheter placement systems include accessing a vasculature with a needle and stabilizing the access site with a guidewire. Once the guidewire is placed, a scalpel may be used to cut or nick the skin and fascia at the insertion site to ease the insertion of the catheter. If the skin nick is not created properly, a skin bridge may form, impeding insertion of the catheter through the skin into the vessel. A skin nicking device may be used to create a repeatable depth of cut, reducing the likelihood of leaving skin bridges around the insertion site. Disclosed herein are advanced catheter placement systems and associated methods for nicking the skin at the insertion site to eliminate skin bridges impeding the insertion of the catheter into the vasculature.

### SUMMARY

Disclosed herein is a catheter placement system including a catheter and a skin nicking device. According to some embodiments, the catheter includes a catheter tube having two or more lumens extending therealong and two or more extension legs, where each of the two or more extension legs is in fluid communication with one of the two or more lumens. The skin nicking device is disposed within one of the two or more lumens, where the skin nicking device includes a blade at a distal end of the skin nicking device, where the blade is configured to nick a skin adjacent an insertion site of the catheter during use to enlarge the insertion site, and further where the blade protrudes from an aperture of the one of the two or more lumens.

In some embodiments, the catheter tube includes a first section having a first outer diameter and a second section having a second outer diameter that is larger than the first outer diameter. According to such embodiments, the catheter tube further includes a transition section extending between the first section and the second section, where the transition section transitions the first outer diameter to the second outer diameter, and where the first section is disposed distal the second section. In some embodiments, the aperture is disposed along the transition section.

In some embodiments, the blade includes a sharp edge directed toward the distal end of the skin nicking device. In some embodiments, the skin nicking device includes a skin nicking device body extending along the catheter tube. In some embodiments, the skin nicking device body extends proximally along the catheter tube and proximally along one of the two or more extension legs such that a proximal end of the skin nicking device exits the one of the two or more extension legs.

In some embodiments, the skin nicking device body is rigid from the proximal end to the distal end. In some embodiments, the skin nicking device body includes one or more rigid portions and one or more flexible portions. In some embodiments, a distal portion of the skin nicking device body is flexible and a proximal portion of the skin nicking device body is rigid.

In some embodiments, the skin nicking device is positionable within the lumen between (i) a distal position, where the blade protrudes from the aperture; and (ii) a proximal position, where the blade is disposed within the lumen proximal the aperture.

In some embodiments, the skin nicking device is rotatable within the lumen between (i) a first orientation, where the blade is directed radially inward; and (ii) a second orientation, where the blade is directed radially outward, thereby enabling the blade to nick the skin.

Also disclosed herein is a method of placing a catheter within a blood vessel of a patient. According to some embodiments, the method includes (i) accessing the blood vessel with a needle to define an insertion site; (ii) inserting a guidewire through the needle and advancing the guidewire along the blood vessel; (iii) removing the needle from the guidewire; (iv) threading a catheter onto the guidewire, where the catheter includes a skin nicking device disposed within a lumen of the catheter, and where the skin nicking device has a blade; (v) nicking the skin adjacent the insertion site to enlarge the insertion site; and (vi) advancing the catheter along the blood vessel.

In some embodiments of the method, nicking the skin adjacent the insertion site includes inserting the catheter through the insertion site.

In some embodiments of the method, the blade protrudes from an aperture of the lumen, and in some embodiments, the method further includes distally displacing the skin nicking device within the lumen to cause the blade to protrude from the aperture

In some embodiments of the method, the skin nicking device includes a skin nicking device body extending along the lumen, and the blade is fixedly attached to the skin nicking device body at a distal end of the skin nicking device body.

In some embodiments of the method, the catheter includes a transition section extending between a distal portion of the catheter defining a first diameter and a proximal portion of the catheter defining a second diameter greater than the first diameter, and the aperture is disposed along the transition section.

In some embodiments, the method further includes proximally displacing the skin nicking device within the lumen to position the blade within the lumen. In some embodiments, the method further includes placing the skin nicking device within the catheter lumen. In some embodiments, the method further includes removing the skin nicking device from the lumen.

Also disclosed herein is a catheter placement device that, according to some embodiments, includes an elongate body defining a proximal end and a distal end and a blade fixedly attached to the elongate body at the distal end, where the blade includes a sharp edge configured to nick a skin of a patient adjacent an insertion site of a catheter to enlarge the insertion site. According to such embodiments, the catheter placement device is configured for placement within a lumen of the catheter, and the blade is configured to protrude from an aperture of the lumen.

In some embodiments, the sharp edge of the blade is directed distally.

In some embodiments, the elongate body includes a lateral slot extending proximally away from the distal end, and the sharp edge is positioned at a bottom of the lateral slot.

In some embodiments, the elongate body includes a flexible distal portion having a preformed curved shape. In some embodiments, the curved shape includes a 180-degree bend. In some embodiments, the sharp edge extends along an outside surface of the curved shape.

In some embodiments, the elongate body includes a longitudinal slit extending proximally away from the distal end, where the longitudinal slit defines a first flexible distal portion and a second distal portion. According to such embodiments, the first flexible distal portion includes a preformed curved shape such that the first flexible distal portion curves away from the second distal portion, and the sharp edge extends along the first flexible distal portion, where the sharp edge is directed inward toward the second distal portion.

These and other features of the concepts provided herein will become more apparent to those of skill in the art in view of the accompanying drawings and following description, which describe particular embodiments of such concepts in greater detail.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system in an unfolded configuration, in accordance with embodiments disclosed herein;

FIG. 1B shows a plan view of a catheter placement system in a folded configuration ready for use, in accordance with embodiments disclosed herein;

FIG. 1C shows a perspective view of a catheter placement system in a folded configuration, in accordance with embodiments disclosed herein;

FIG. 2 shows a side view of a catheter of a catheter placement system in an unfolded configuration, in accordance with embodiments disclosed herein;

FIG. 3A shows close up detail of a distal portion of the catheter of FIG. 2, in accordance with embodiments disclosed herein;

FIGS. 3B-3C show cross-section views of the catheter of FIG. 3A, in accordance with embodiments disclosed herein;

FIG. 4 shows a longitudinal cross-section view of a distal portion of a catheter placement system, in accordance with embodiments disclosed herein;

FIGS. 5A-5E show an exemplary method of use for a catheter placement system, in accordance with embodiments disclosed herein;

FIGS. 6A-6B illustrate cross sectional views of a skin nicking device, in accordance with some embodiments;

FIG. 6C illustrates a cross sectional view of a catheter, in accordance with some embodiments;

FIGS. 6D-6G illustrate additional embodiments of the skin nicking device, in accordance with some embodiments;

FIGS. 7A-7C are various views of the catheter placement system illustrating an exemplary method of placing a catheter using the system including the skin nicking device, in accordance with some embodiments; and

FIG. 8 illustrates a flow chart of an exemplary method of placing a catheter using the catheter placement system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

The phrases “connected to,” “coupled to,” and “in communication with” refer to any form of interaction between two or more entities, including but not limited to mechanical, electrical, magnetic, electromagnetic, fluid, and thermal interaction. Two components may be coupled to each other even though they are not in direct contact with each other. For example, two components may be coupled to each other through an intermediate component.

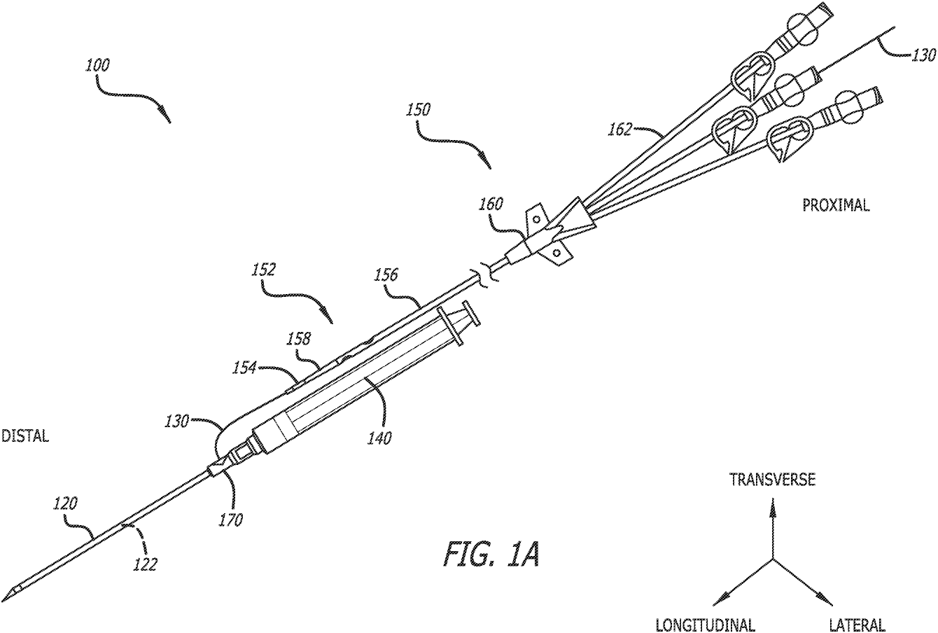

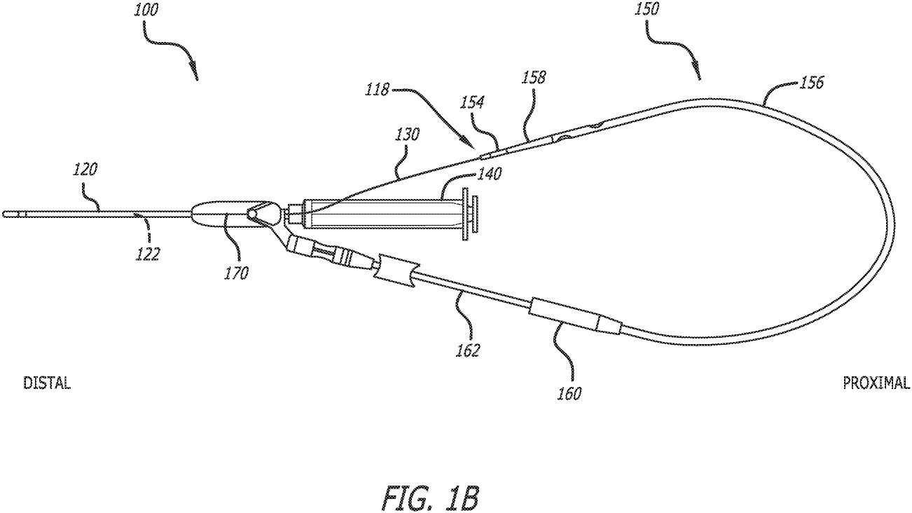

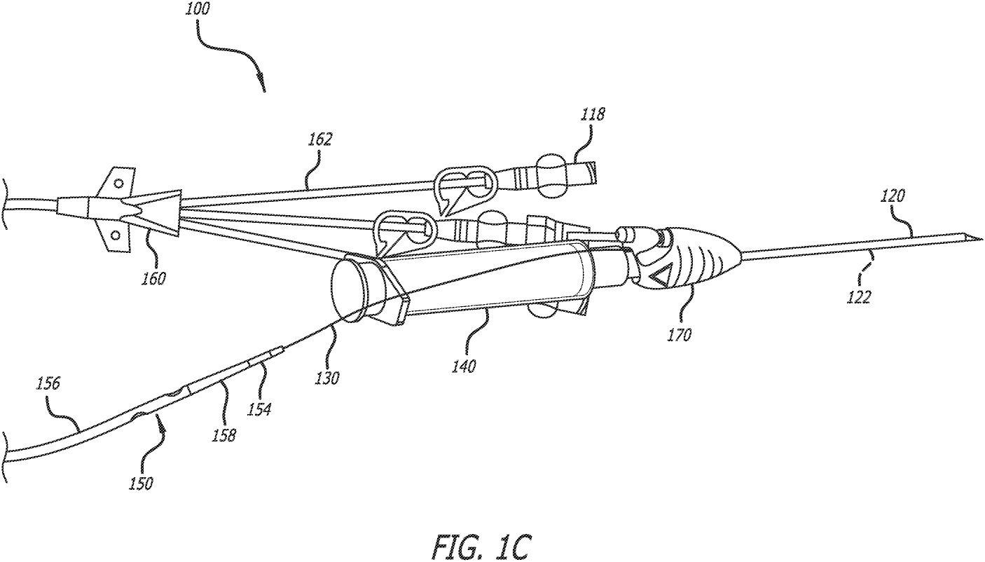

FIGS. 1A-1C show an exemplary catheter placement system (“system”) 100, generally including a needle 120 having a needle housing (“housing”) 170, a guidewire 130, a syringe system 140, and a catheter 150. FIG. 1A shows the system 100 in an unfolded configuration for ease of illustration. FIG. 1B shows a plan view of the system 100 in a folded configuration ready for use. FIG. 1C shows a perspective view the system 100 in a folded configuration. In an embodiment the catheter placement system 100 can be a Rapidly Insertable Central Catheter (RICC) placement system 100 that is configured to place an RICC 150. However, it will be appreciated that other catheter placement systems configured to place other types of catheters are also contemplated. Exemplary other types of catheters can include peripheral intravenous (PIV) catheters, peripherally inserted central catheter (PICC), central venous catheters (CVC), midline catheters, dialysis catheters, single lumen catheters, multi-lumen catheters, or the like.

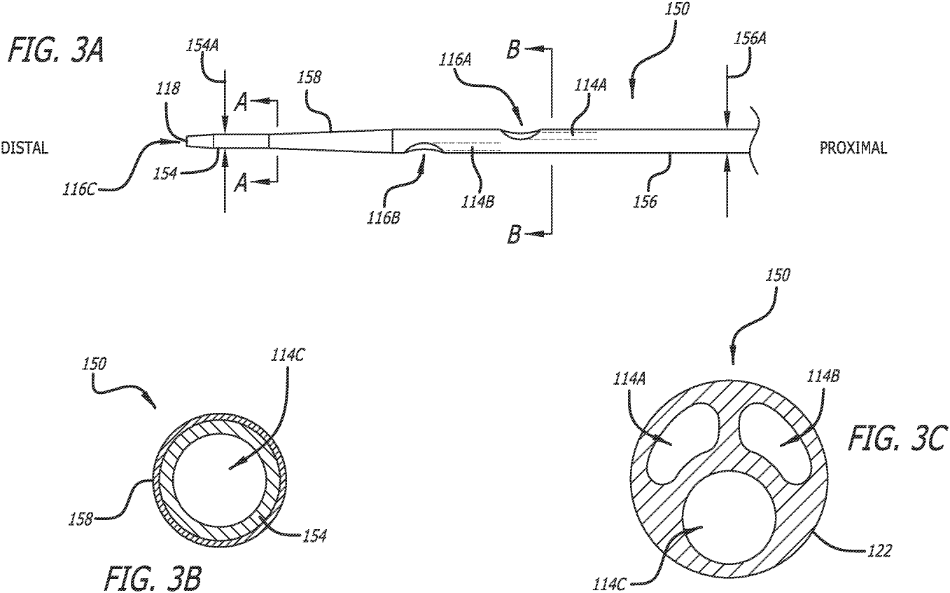

In an embodiment, the catheter 150 can generally include a catheter body (or tube) 152 coupled with a catheter hub (“hub”) 160 a proximal end of the catheter body 152. The catheter 150 further includes one or more extension legs 162 (e.g., three extension legs 162 as shown) extending proximally from the hub 160. Each extension leg of the one or more extension legs 162 can be in fluid communication with a lumen of the catheter body 152. In the illustrated embodiment, the catheter body 152 may include a first section 154 extending proximally away from a distal end 118 of the catheter body 152 and a second section 156 extending distally away from the hub 160. A transition section 158 is disposed between the first section 154 and the second section 156. The first section 154 can include a single lumen and may define a first outer diameter 154A (see FIG. 3A), the second section 156 can include two or more lumens and may define a second diameter 155A (see FIG. 3A) that is larger than the first diameter 154A. The transition section 158 disposed between the first section 154 and the second section 156 can define a tapered shape to transition the first diameter to the second diameter. A guidewire 130 can extend through a lumen of the catheter 150 from a proximal end of an extension leg 162 to a distal end 118 of the catheter body 152.

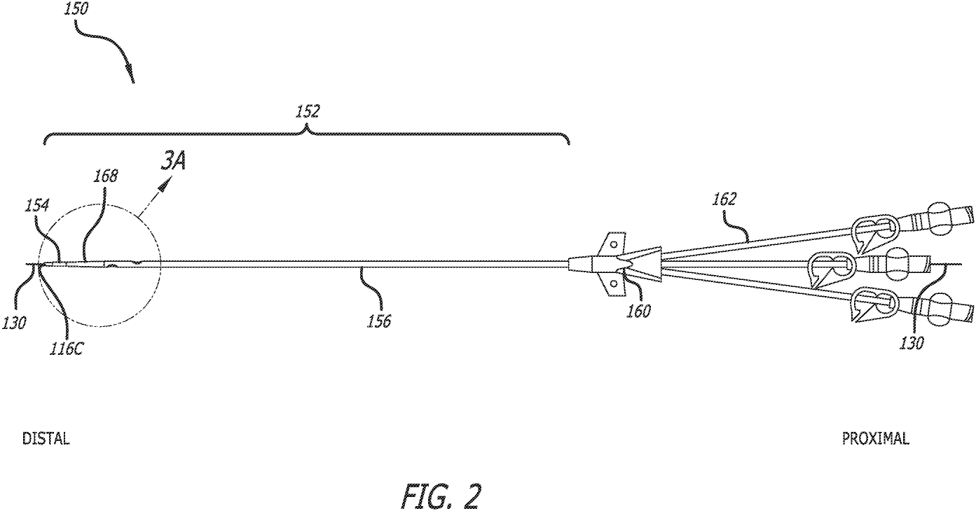

FIG. 2 shows further details of the catheter 150 of the system 100. As described herein, different sections of the catheter 150 may be configured to perform different functions and as such may include different mechanical properties. For example, the first section 154 and the transition section 158 may be more rigid than the second section 156. In some embodiments, the first and transition sections 154, 158 may be formed of harder durometer material relative to the second section 156. As such, the first section 154 and transition section 158 can withstand greater axial forces that the second section 156 without kinking, buckling, or collapsing during use. The second section 156 can be formed of a softer durometer, or a more compliant material to facilitate advancing the second section 156 through tortuous vascular pathways of the vasculature.

FIGS. 3A-3C show further details of a distal portion of the catheter 150, including the first section 154, the second section 156, and the transition section 158. In an embodiment, the second section 156 can include a proximal lumen 114A terminating at a proximal lumen aperture 116A, and a medial lumen 114B terminating at a medial lumen aperture 116B. In some embodiments, each of the proximal lumen aperture 116A and the medial lumen aperture 116B can extend through a side wall of the second section 156. One or both of the proximal lumen aperture 116A and the medial lumen aperture 116B can be disposed along the transition section 158. In some embodiments, the proximal lumen aperture 116A can be disposed proximal the medial lumen aperture 116B.

FIG. 3B shows a cross-section end view of the catheter body 152 at point “A” of FIG. 3A. As shown, the first section 154 can define a single lumen and the first outer diameter 154A. A distal lumen 114C of the catheter 150 can extend to the distal end 118 of the catheter 150 defining a distal lumen aperture 116C (FIG. 3A). FIG. 3C shows a cross-section end view of the second section 156 at point “B” of FIG. 3A, showing the proximal lumen 114A, medial lumen 114B, and distal lumen 114C. Each of the proximal lumen 114A, the medial lumen 114B, and distal lumen 114C may be in fluid communication with one or more of the extension legs 162.

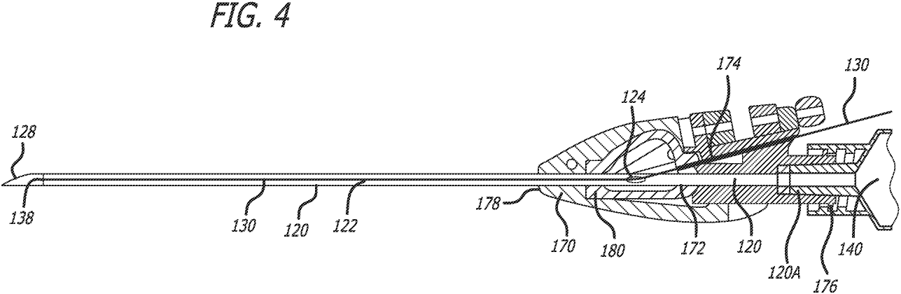

FIG. 4 shows a longitudinal cross-sectional view of a distal portion of a catheter placement system 100 including the needle 120, the guidewire 130, a distal portion of the syringe system 140, and the needle housing (“housing”) 170. A needle splitter system 180 is also shown as described in more detail herein. The needle 120 may include a needle hub 120A at a proximal end of the needle 120, where the needle hub 120 can be coupled to, and supported by, a distal end of the syringe system 140. The syringe system 140 can be in fluid communication with needle lumen 122. The syringe system 140 can be configured to draw fluid proximally through the needle lumen 122. In an embodiment, the needle 120 can include a guidewire aperture 124 extending through a wall of the needle 120 to the needle lumen 122. The guidewire 130 can be inserted through the guidewire aperture 124 and advanced along the needle lumen 122. In an embodiment, a distal tip 138 of the guidewire 130 can be positioned proximate a distal tip 128 of the needle 120. As such, when the distal tip 128 of the needle 120 is disposed with the vasculature, the distal tip 138 of the guidewire 130 is also disposed within the vasculature.

The housing 170 can include a housing lumen 172 extending between a proximal end 176 and a distal end 178 of the housing 170, where the housing lumen 172 is configured to slidably receive the needle 120 therethrough. The housing 170 can further include a guidewire lumen 174 communicating with the housing lumen 172 and extending at an angle therefrom. Further, the proximal end 176 of the housing can releasably engage one or both of a needle hub 120A and a distal portion of the syringe system 140. In the illustrated embodiment, when the housing 170 is coupled with the syringe system 140, the guidewire aperture 124 of the needle 120 may be disposed in alignment with the guidewire lumen 174 of the housing 170. As such, the guidewire 130 may be inserted through the guidewire lumen 174 of the housing 170, through the guidewire aperture 124 of the needle 120, and into the needle lumen 122.

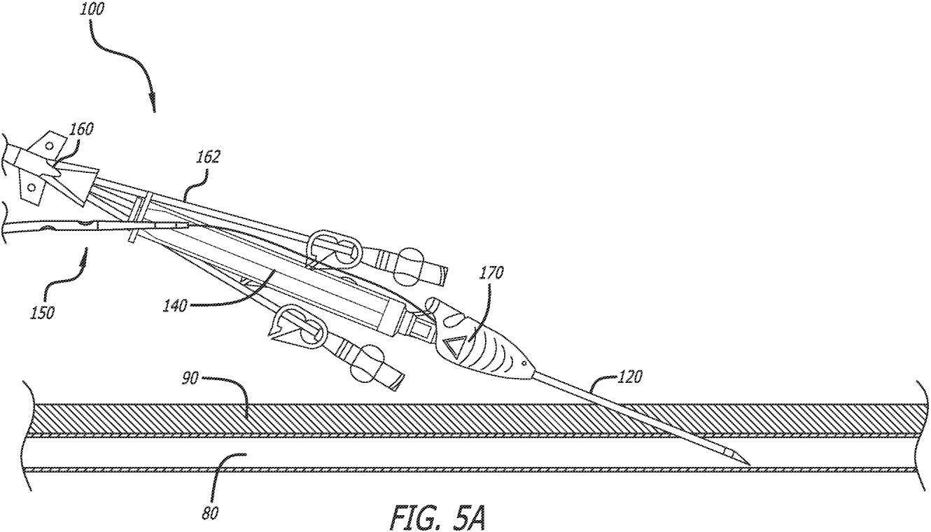

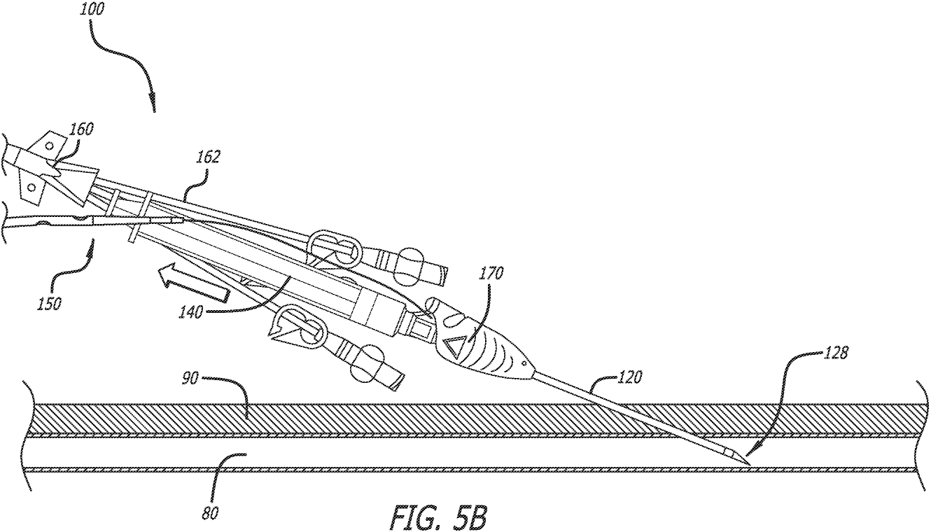

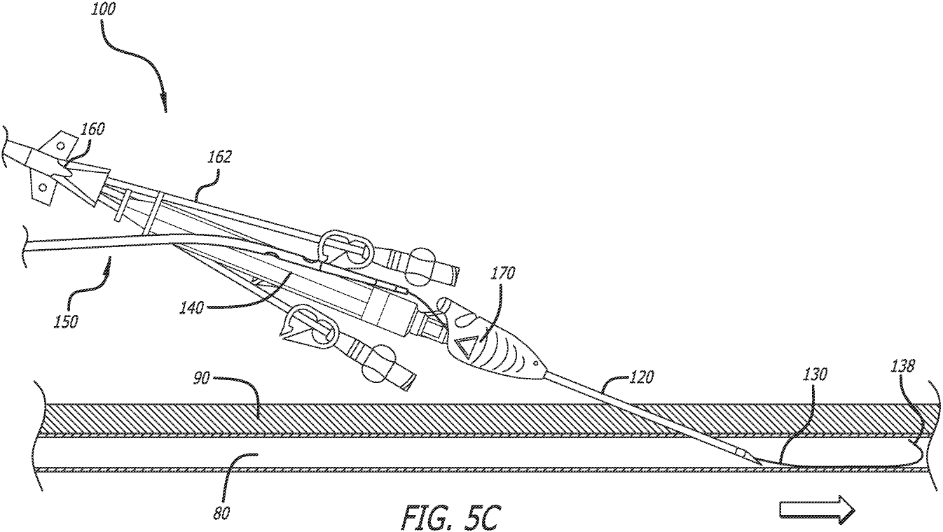

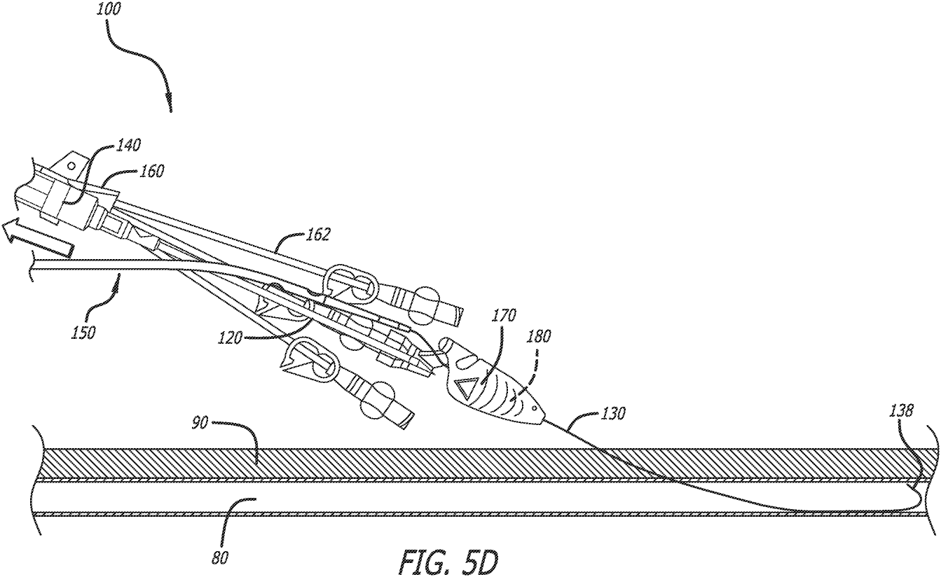

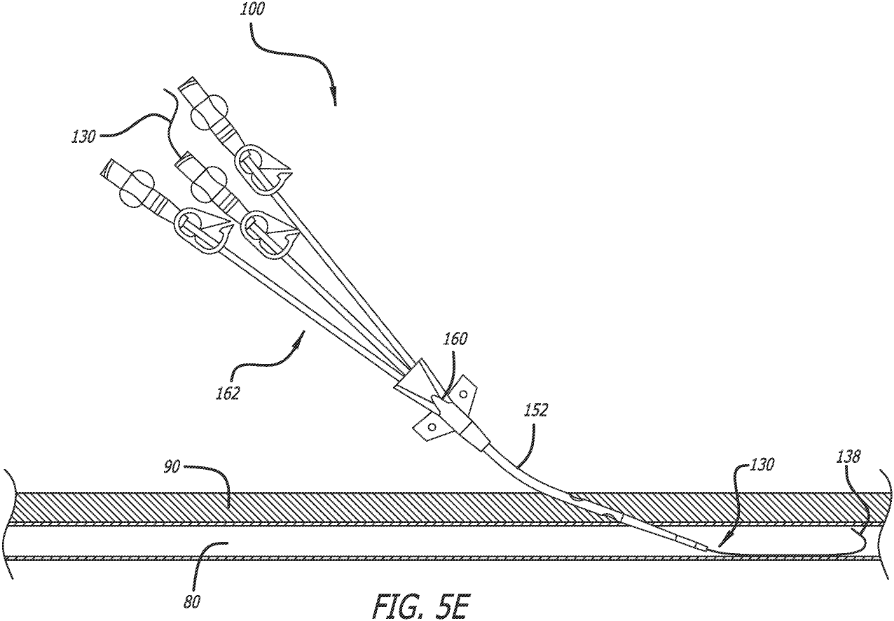

FIGS. 5A-5E show various views of the system 100 depicting an exemplary method of placing a catheter 150 with in a blood vessel using the catheter placement system 100. As shown in FIG. 5A, the needle 120 can penetrate surface tissues 90 (e.g., skin) of the patient and access a vasculature 80, forming an insertion site. As shown in FIG. 5B, a syringe system 140, or similar device can draw a fluid (e.g., blood) proximally through a needle lumen 122. A user can observe a color of the fluid and/or pulsatile flow characteristics and, thereby confirm that the distal tip 128 is disposed within the vasculature 80. Where incorrect vascular access is confirmed, the needle 120 can be withdrawn and the insertion site can be closed. As shown in FIG. 5C, once presence of the distal tip 128 with the vasculature 80 has been confirmed, the guidewire 130 can then be advanced through the needle lumen 122 and into the vasculature 80.

As shown in FIG. 5D, the needle 120 and syringe system 140 assembly can be laterally separated from the guidewire 130 while leaving a distal portion of the guidewire 130 in place within the vasculature 80. As described in more detail herein, the housing 170 can include a splitter system 180 configured to split the needle 120 longitudinally. A portion of the guidewire 130 can pass between the two halves of the needle 120 to enable lateral separation of the needle 120 from the guidewire 130.

As shown in FIG. 5E, with the needle 120 and syringe system 140 assembly disengaged from the guidewire 130, the catheter 150 can then be advanced over the guidewire 130 and into the vasculature. The first section 154 of the catheter 150, having only a single lumen and defining the first outer diameter 154A, can enter the vasculature 80 through the insertion site and over the guidewire 130. The catheter 150 be displaced to insert the transition section 158 through the insertion site, where the transition section 158 dilates the insertion site to allow the second section 156 having the second outer diameter 156A to enter the vasculature 80. Once the catheter 150 has been placed with the vasculature 80, the guidewire 130 may be withdrawn proximally.

Further details and embodiments of such catheter placement systems 100 can be found, for example, in US 10,376,675, US 2019/0255294, US 2021/0069471, US 2021/0085927, US 2021/0113809, US 2021/0113810, US 2021/0121661, US 2021/0121667, US 2021/0228843, US 2021/0322729, US 2021/0330941, US 2021/0330942, US 2021/0361915, US 2021/0402153, US 2021/0402149, US 2022/0001138, US 2022/0032013, and U.S. Pat. Application No. 17/882,388 filed Aug. 5, 2022, each of which is incorporated by reference in its entirety into this application.

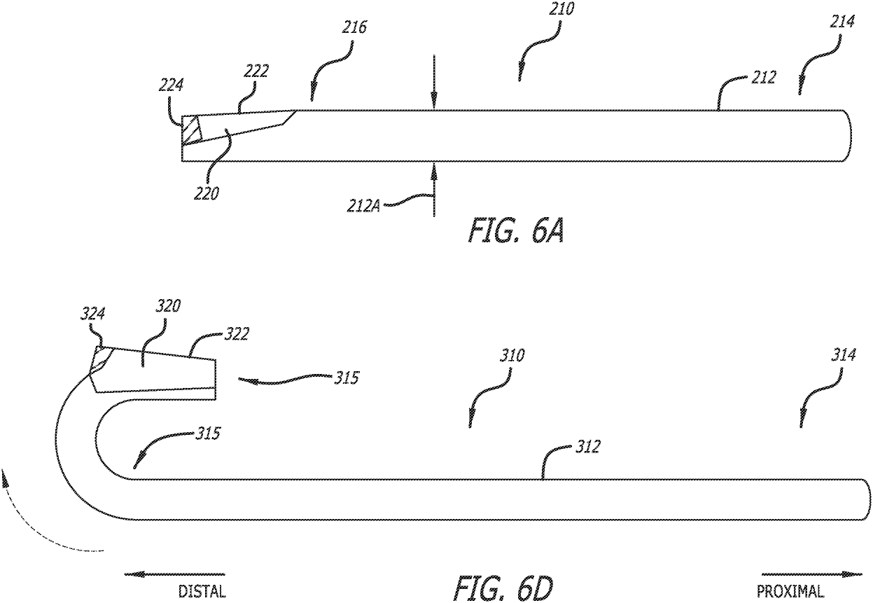

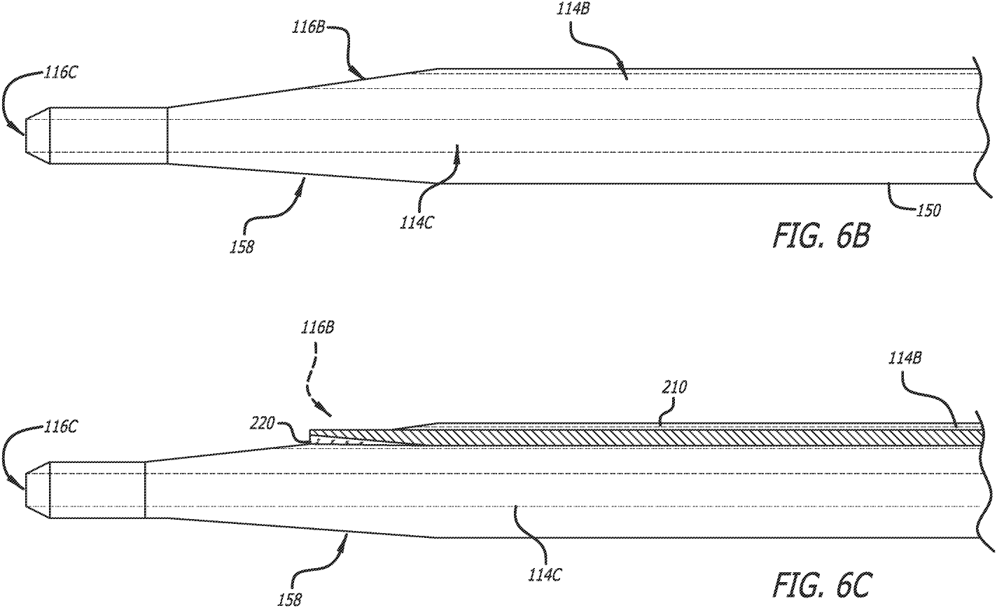

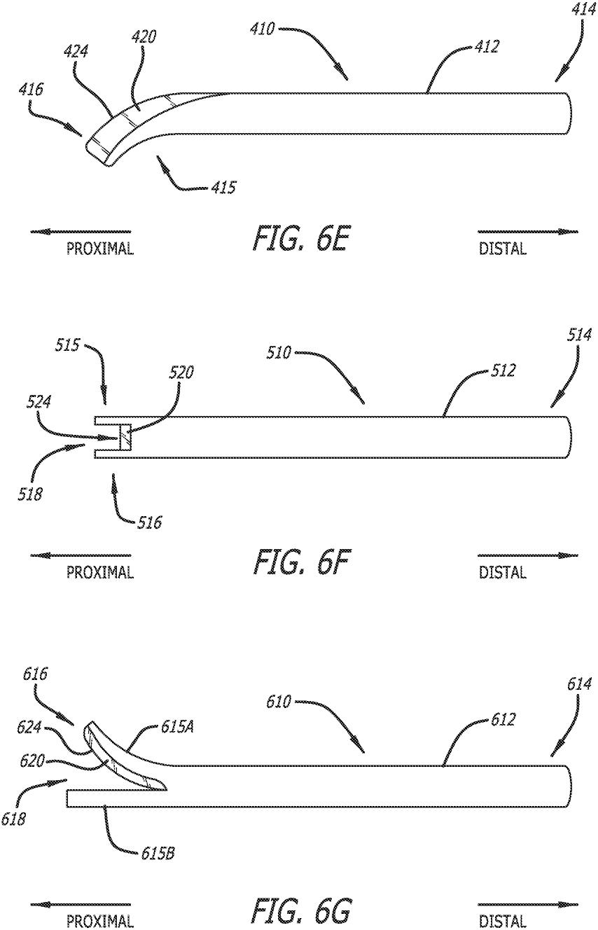

In some instances, it may be beneficial to enlarge the insertion to accommodate the catheter 150. FIG. 6A illustrates a side view of a skin nicking device 210, in accordance with some embodiments. In some embodiments, the skin nicking device 210 may be coupled with or integrated into the catheter placement system 100. In some embodiments, the skin nicking device 210 may be a catheter placement device and used with the catheter placement system 100 to place the catheter 150. As illustrated in FIG. 6A, the skin nicking device 210 may include a skin nicking device body 212 defining a proximal end 214 and a distal end 216. In some embodiments, the skin nicking device body 212 may include a stiffening stylet. In some embodiments, the skin nicking device 210 may be inserted into a lumen of the catheter 150, such as the medial lumen 114B of the catheter 150, for example. In the illustrated embodiment, the skin nicking device 210 includes a blade 220 defining a sharp edge 224, where the blade 220 is disposed adjacent the distal end 216. In other embodiments, the skin nicking device 210 may include more than one blade 220. The blade 220 is generally configured to enable nicking or cutting of the surface tissue 90 (e.g., skin) via the sharp edge 224. In some embodiments, the skin nicking device body 212 may be rigid from the proximal end 214 to the distal end 216. For example, a portion of the skin nicking device body 212 adjacent the distal end 216 may be more flexible in relation to a portion of the skin nicking device body 212 adjacent the proximal end 214. In some embodiments, the blade 220 may also include a dull edge 222. In some embodiments, the sharp edge 224 may be oriented in direction toward the distal end 216 while the dull edge 222 may oriented in a different direction, such as toward the proximal end 214, for example. The skin nicking device body 212 may generally define a body diameter 212A. In the illustrated embodiment, the sharp edge 224 may be disposed inward of the body diameter 212A so that the sharp edge 224 is constrained away from an inside surface of the catheter lumen, so as to prevent cutting the catheter 150. In other embodiments, the sharp edge 224 may extend radially beyond of the body diameter 212A. In some embodiments, the proximal end 214 may include an attachment member (not shown) configured to detachably couple the skin nicking device 210 to the catheter 150 in order to properly place the skin nicking device 210 within the catheter 150 as the catheter 150 is inserted into the vasculature 80.

FIG. 6B illustrates a cross-sectional detailed side view of a distal portion of the catheter 150, in accordance with some embodiments. As stated above, the catheter 150 may include the proximal lumen 114A (see FIG. 3A), the medial lumen 114B, and the distal lumen 114C. The distal lumen 114C includes a distal lumen aperture 116C, and the medial lumen 114B includes a medial lumen aperture 116B, where the medial lumen aperture 116B is a side aperture disposed proximal the distal lumen aperture 116C. Any or all to the proximal lumen 114A, the medial lumen 114B, and the distal lumen 114C may be configured for placement of the skin nicking device 210 therein. In the illustrated embodiment, the medial lumen aperture 116B may be disposed along the transition section 158.

FIG. 6C illustrates the cross-sectional detailed side view of FIG. 6B coupled with the skin nicking device 210, in accordance with some embodiments. As shown in FIG. 6C, in the illustrated embodiment, the skin nicking device 210 may be inserted into the medial lumen 114B and advanced through the medial lumen 114B until the distal end 216 of the skin nicking device 210 including the blade 220 extends out of the medial lumen aperture 116B. In some embodiments, the skin nicking device 210 may be rotated axially within the medial lumen 114B to orient the blade 220 so that the blade is directed radially outward from the catheter 150, i.e., oriented to facilitate nicking of the skin. In some embodiments, when inserting the skin nicking device 210 into the medial lumen 114B, the skin nicking device 210 may be oriented with the blade 220 directed towards the distal lumen 114C, as shown in FIG. 6C. In some embodiments, when inserting the skin nicking device 210 into the medial lumen 114B, the skin nicking device 210 may be oriented with the blade 220 directed radially outward, i.e., in the opposite direction shown. In some embodiments, wherein the distal end 216 extends from the medial lumen aperture 116B, the distal end 216 may be configured to extend parallel with the catheter 150, e.g., sit flush against the transition section 158 to create a repeatable depth of skin nick and reduce the likelihood of creation of a skin bridge during placement of the catheter 150.

Although not shown, the skin nicking device 210 (or more specifically, the skin nicking device body 212) may extend proximally along the medial lumen 114B and may further extend along a corresponding extension leg 162 (see FIG. 2) such that the proximal end 214 extends beyond (i.e., exits) the corresponding extension leg 162. During use, a user may grasp the proximal end 214 and manipulate the skin nicking device 210 thereby. For example, the user may longitudinally displace the skin nicking device 210 within the medial lumen 114B. In some embodiments, the user may displace the skin nicking device 210 between (i) a distal position, where the blade 220 protrudes from the medial lumen aperture 116B (FIG. 6C) and (ii) a proximal position (not shown), where the blade 220 is disposed within the medial lumen 114B proximal the medial lumen aperture 116B. Similarly, in some embodiments, the user may rotate the skin nicking device 210 within the medial lumen 114B. In some embodiments, the user may rotate the skin nicking device 210 between (i) a first orientation, where the blade 220 is directed radially inward (FIG. 6C) and (ii) a second orientation (not shown), where the blade 220 is directed radially outward, thereby enabling the blade 220 to nick the skin.

FIG. 6D illustrates a second embodiment of a skin nicking device 310 that can, in certain respects, resemble components of the skin nicking device 210 described in connection with FIG. 6A. It will be appreciated that all the illustrated embodiments may have analogous features. Accordingly, like features are designated with like reference numerals, with the leading digits incremented to “3.” For instance, the skin nicking device body is designated as “212” in FIG. 6A, and an analogous skin nicking device body is designated as “312” in FIG. 6D. Relevant disclosure set forth above regarding similarly identified features thus may not be repeated hereafter. Moreover, specific features of the skin nicking device 210 and related components shown in FIG. 6A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the skin nicking device 310 of FIG. 6D. Any suitable combination of the features, and variations of the same, described with respect to the skin nicking device 210 and components illustrated in FIG. 6A can be employed with the skin nicking device 310 and components of FIG. 6D, and vice versa. This pattern of disclosure applies equally to further embodiments depicted in subsequent figures and described hereafter.

The skin nicking device 310 includes a skin nicking device body 312 where a distal portion 315 is flexible in bending. For example, the distal portion 315 may be flexible while the balance of the skin nicking device body 312 extending distally away from the proximal end 314 may be rigid. In some embodiments, the distal end portion 315 may include a preformed shape, such as the preformed curved shape shown in FIG. 6D, for example. In some embodiment, the preformed shape may include about a 180-degree bend such that the distal end 316 is directed toward the proximal end 314. The structure of the skin nicking device body 312 may generally be configured so that the distal portion 315 (i) is constrained in a straight shape (or the shape of the catheter 150) when the skin nicking device 310 is disposed within the medial lumen 114B, and (ii) resumes the preformed shape when the distal portion 315 extends from the medial aperture 116B. In the illustrated embodiment, the sharp edge 324 of the blade 320 is directed opposite the distal end 316 so that the sharp edge 324 is directed distally as a result of 180-degree bend. Similarly, the sharp edge 324 may be directed proximally when the distal portion 315 is constrained toward the straight shape. In some embodiments, the distal end 316 being curved back towards the proximal end 314 may allow the distal end 316 including the blade 320 to extend out of the medial lumen aperture 116B while the rest of the skin nicking device body 312 resides within the medial lumen 114B.

FIG. 6E illustrates a third embodiment of a skin nicking device 410 that includes a skin nicking device body 412 where a distal portion 415 is flexible in bending. For example, the distal portion 415 may be flexible while the balance of the skin nicking device body 412 extending distally away from the proximal end 414 may be rigid. In some embodiments, the distal end portion 415 may include a preformed shape, such as the preformed curved shape shown in FIG. 6E, for example. The structure of the skin nicking device body 412 may be generally configured so that the distal portion 415 (i) is constrained in a straight shape (or the shape of the catheter 150) when the skin nicking device 410 is disposed within the medial lumen 114B, and (ii) resumes the preformed curved shape when the distal portion 415 extends from the medial lumen aperture 116B. In the illustrated embodiment, the sharp edge 424 of the blade 420 is directed away from an outside surface of the curve.

FIG. 6F illustrates a fourth embodiment of a skin nicking device 510 that includes a skin nicking device body 512 where a distal end 516 includes a lateral slot 518 extending proximally away from the distal end 516. A blade 520 is disposed at the bottom of the lateral slot 518 so that the sharp edge 524 is directed distally. In use, the lateral slot 518 captures the skin or tissue so that the sharp edge 524 may cut the skin or tissue as the skin nicking device 510 is displaced distally.

FIG. 6G illustrates a fifth embodiment of a skin nicking device 610 the includes a skin nicking device body 612 having a longitudinal slit 618 extending proximally away from the distal end 616, where the longitudinal slit 618 defines first and second distal portions 615A, 615B. The first distal portion 615A is flexible in bending and defines a preformed curved shape, where the first distal portion 615A curves away from the second distal portion 615B, as shown in FIG. 6G. The structure of the skin nicking device body 612 may generally be configured so that the first distal portion 615A (i) is constrained in a straight shape (or the shape of the catheter 150) when the skin nicking device 610 is disposed within the medial lumen 114B, and (ii) resumes the preformed curved shape when the first distal portion 615A extends from the medial lumen aperture 116B. In the illustrated embodiment, the sharp edge 624 of the blade 620 is directed radially inward. In use, the skin or tissue is captured between the first and second distal portions 615A, 615B so that the sharp edge 624 may cut the skin or tissue as the skin nicking device 610 is displaced distally.

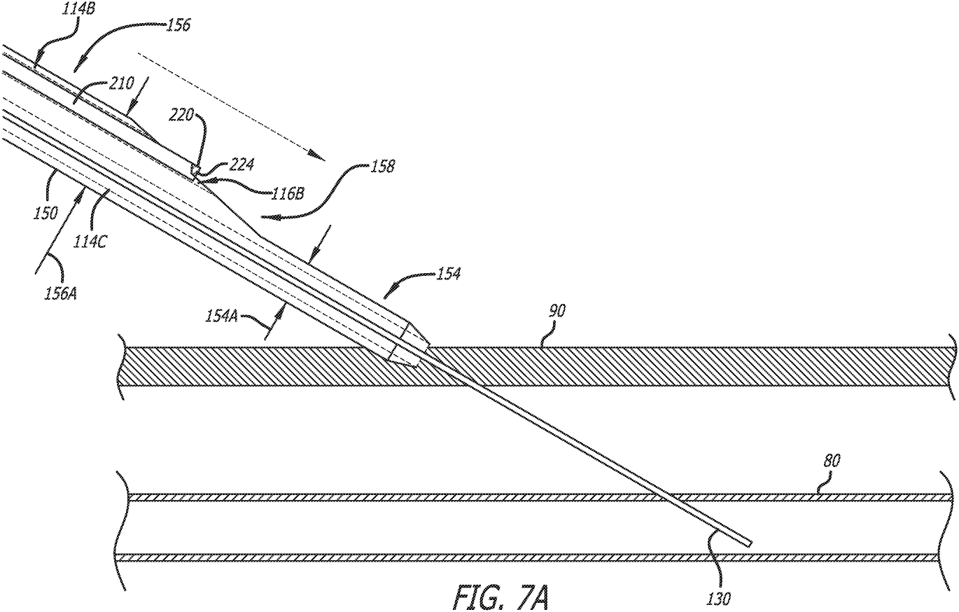

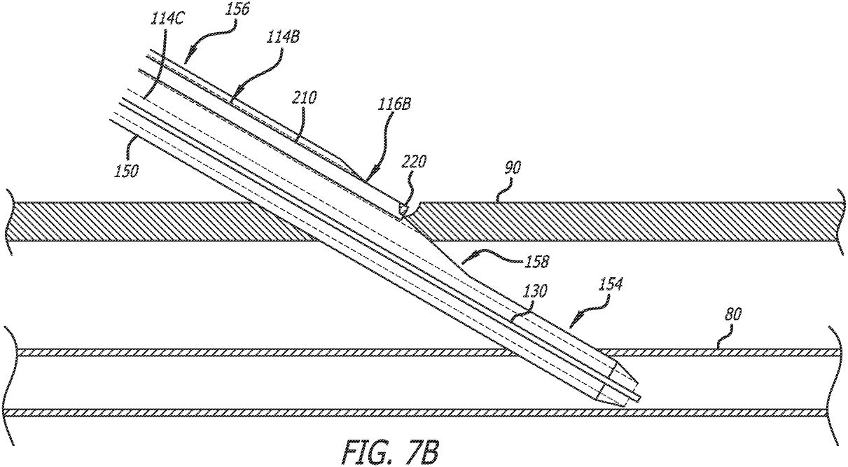

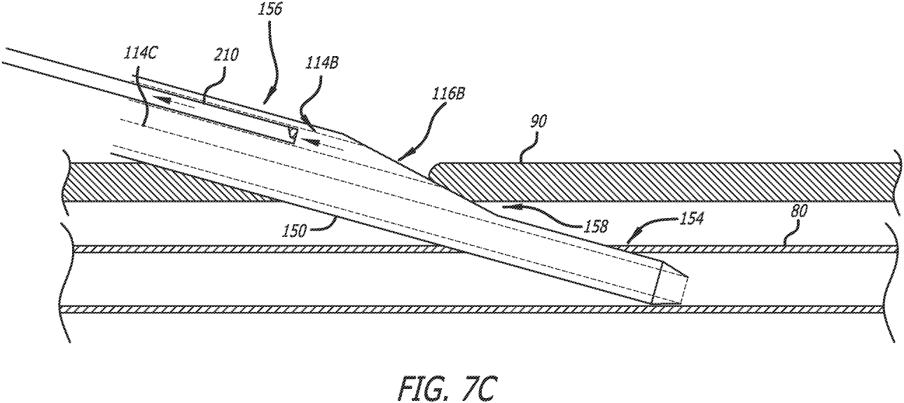

FIGS. 7A-7C illustrate various a cross-sectional views of the system 100 depicting an exemplary method of placing the catheter 150 using the catheter placement system 100 including the skin nicking device 210, in accordance with some embodiments. In some embodiments, in placing the catheter 150, the introducer needle 120 has accessed the vasculature 80 with the guidewire 130 being threaded through the needle 120 into the vasculature 80. The introducer needle 120 has been removed from the vasculature 80, leaving the distal portion of guidewire 130 within the vasculature 80 (see FIGS. 5C-5D). As illustrated in FIG. 7A, the skin nicking device 210 may be placed within the medial lumen 114B of the catheter 150 and extended through the medial lumen aperture 116B. The catheter 150 may include the transition section 158 tapering from the smaller first outer diameter 154A of the first section 154 to the larger second diameter 156A of the second section 156. The guidewire 130 may be threaded through the distal lumen 114C and the catheter 150 may be slid over (or advanced along) the guidewire 130 toward the insertion site.

As illustrated in FIG. 7B, the transition section 158 may dilate the insertion site as the catheter 150 is slid over the guidewire 130, forcing the skin and surface tissue 90 around the insertion site to be taut. The skin nicking device 210 having the blade 220 extending out of the medial lumen aperture 116B may nick the skin and nearby tissue 90 at the insertion site, thereby enlarging the insertion site while reducing the likelihood of creating a skin bridge. Advantageously, the skin nicking device 210 not only nicks the skin and surface tissues 90 and provides an incision appropriately sized for the catheter 150 but provides the incision on the same pathway as the catheter 150. As illustrated in FIG. 7C, once the skin has been nicked, the skin nicking device 210 may be proximally displaced within the medial lumen 114B so that the blade 220 is disposed within the medial lumen 114B, thereby preventing the blade 220 from further cutting tissue. Thereafter, the catheter 150 may be fully inserted into the vasculature 80. In some embodiments, the skin nicking device 210 may be removed from the medial lumen 114B.

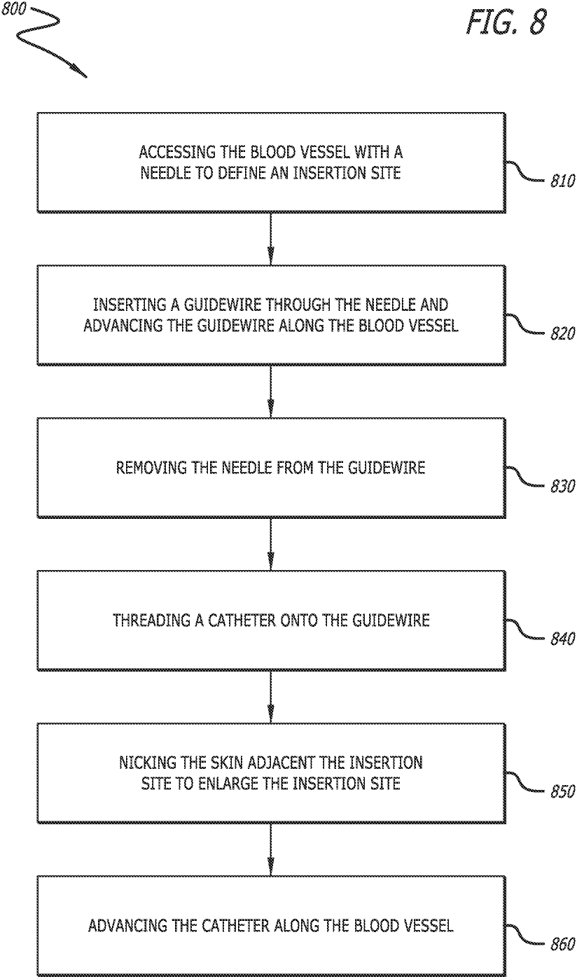

FIG. 8 illustrates a flow chart of an exemplary method 800 of placing a catheter 150 using the catheter placement system 100 that, in accordance with some embodiments, may include all or any subset of the following steps, actions or processes. The method 800 may include accessing the blood vessel with a needle to define an insertion site (block 810)

The method 800 may further include (i) inserting a guidewire through the needle and advancing the guidewire along the blood vessel (block 820) and removing the needle from the guidewire (block 830).

The method 800 may further include threading a catheter onto the guidewire (block 840) where the catheter includes the skin nicking device disposed within a lumen of the catheter. In some embodiments of the method 800, the blade protrudes from an aperture of the lumen, and in some embodiments, the method 800 further includes distally displacing the skin nicking device within the lumen to cause the blade to protrude from the aperture. In some embodiments of the method 800, the skin nicking device includes a skin nicking device body extending along the lumen, and the blade is fixedly attached to the skin nicking device body at a distal end of the skin nicking device body. In some embodiments, the method 800 may further include proximally displacing the skin nicking device within the lumen to position the skin nicking device within the lumen so that the blade protrudes from the aperture. In some embodiments, the method 800 may further include rotating the skin nicking device within the lumen to orient the blade to directed radially outward. In some embodiments, the method 800, the catheter and the skin nicking device may be provided separately. As such, the method 800 may further include initially placing the skin nicking device within the catheter lumen.

The method 800 may further include nicking the skin adjacent the insertion site to enlarge the insertion site (block 850). In some embodiments of the method 800, nicking the skin may take place during insertion or a result of insertion of the catheter through the insertion site. In some embodiments of the method 800, the catheter includes a transition section extending between a distal portion of the catheter defining a first diameter and a proximal portion of the catheter defining a second diameter greater than the first diameter, and the aperture is disposed along the transition section.

The method 800 may further include advancing the catheter along the blood vessel (block 860) to complete the catheter placement. In some embodiments, the method 800 may further include removing the skin nicking device from the lumen.

In some embodiments, the method 800 may include detecting a target vessel includes using one or more medical devices to detect the target vessel within the vasculature. In some embodiments, the one or more medical device may include an ultrasound probe.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter placement system, comprising:
a catheter, comprising:
a catheter tube having two or more lumens extending therealong; and
two or more extension legs, each of the two or more extension legs in fluid communication with one of the two or more lumens; and
a skin nicking device disposed within one of the two or more lumens, wherein:
the skin nicking device includes a blade at a distal end of the skin nicking device, the blade configured to nick a skin adjacent an insertion site of the catheter during use to enlarge the insertion site, and
the blade protrudes from an aperture of the one of the two or more lumens.

2. The system according to claim 1, wherein:
the catheter tube includes:
a first section having a first outer diameter;
a second section having a second outer diameter larger than the first outer diameter; and
a transition section extending between the first section and the second section,
the transition section is configured to transition the first outer diameter to the second outer diameter, and
the first section is disposed distal the second section.

3. The system according to claim 2, wherein the aperture is disposed along the transition section.

4. The system according to claim 1, wherein the blade includes a sharp edge directed toward the distal end of the skin nicking device.

5. The system according to claim 1, wherein the skin nicking device includes a skin nicking device body extending along the catheter tube.

6. The system according to claim 5, wherein the skin nicking device body extends proximally along the catheter tube and proximally along one of the two or more extension legs such that a proximal end of the skin nicking device exits the one of the two or more extension legs.

7. The system according to claim 5, wherein the skin nicking device body is rigid from the proximal end to the distal end.

8. The system according to claim 5, wherein the skin nicking device body includes one or more rigid portions and one or more flexible portions.

9. The system according to claim 8, wherein a distal portion of the skin nicking device body is flexible and a proximal portion of the skin nicking device body is rigid.

10. The system according to claim 1, wherein the skin nicking device is positionable within the lumen between:
a distal position, where the blade protrudes from the aperture; and
a proximal position, where the blade is disposed within the lumen proximal the aperture.

11. The system according to claim 1, wherein the skin nicking device is rotatable within the lumen between:
a first orientation, where the blade is directed radially inward; and
a second orientation, where the blade is directed radially outward, thereby enabling the blade to nick the skin.

12-20. (canceled)

21. A catheter placement device, comprising:
an elongate body defining a proximal end and a distal end;
a blade fixedly attached to the elongate body at the distal end, the blade including a sharp edge configured to nick a skin of a patient adjacent an insertion site of a catheter to enlarge the insertion site, wherein:
the catheter placement device is configured for placement within a lumen of the catheter, and
the blade is configured to protrude from an aperture of the lumen.

22. The device according to claim 21, the sharp edge of the blade is directed distally.

23. The device according to claim 21, wherein:
the elongate body includes a lateral slot extending proximally away from the distal end, and
the sharp edge is positioned at a bottom of the lateral slot.

24. The device according to claim 21, wherein the elongate body includes a flexible distal portion having a preformed curved shape.

25. The device according to claim 24, wherein the curved shape includes a 180-degree bend.

26. The device according to claim 24, wherein the sharp edge extends along an outside surface of the curved shape.

27. The device according to claim 21, the elongate body includes a longitudinal slit extending proximally away from the distal end, the longitudinal slit defining a first flexible distal portion and a second distal portion, wherein:
the first flexible distal portion includes a preformed curved shape such that the first flexible distal portion curves away from the second distal portion, and
the sharp edge extends along the first flexible distal portion, the sharp edge directed inward toward the second distal portion.
